(19) United States
(12) Patent Application Publication
(10) Pub. No.: US 2021/0362003 A1
(43) Pub. Date: Nov. 25, 2021

(54) INTERACTIVE UPPER LIMB REHABILITATION TRAINING SYSTEM

(71) Applicant: SOUTH CHINA UNIVERSITY OF TECHNOLOGY, Guangdong (CN)

(72) Inventors: Longhan XIE, Guangdong (CN); Siqi CAI, Guangdong (CN); Shuangyuan HUANG, Guangdong (CN); Guofeng Li, Guangdong (CN)

(73) Assignee: SOUTH CHINA UNIVERSITY OF TECHNOLOGY, Guangdong (CN)

(21) Appl. No.: 16/630,460

(22) PCT Filed: Dec. 28, 2018
(86) PCT No.: PCT/CN2018/124825

§ 371 Date: Jan. 12, 2020

(30) Foreign Application Priority Data
Nov. 14, 2018 (CN) 201811354114.0

(51) Int. Cl. A63B 23/14; A63B 23/12; A63B 71/06; G16H 50/30

(52) U.S. Cl. CPC A63B 23/14; A63B 23/1281; A63B 2220/16; G16H 50/30; A63B 71/0622

(57) ABSTRACT

An interactive upper limb rehabilitation training system includes an interactive display screen, a host computer control center, a dual-arm rehabilitation robot, a movable space adjustable dual-arm robot base, and a position tracker. The dual-arm rehabilitation robot is mounted on the movable space adjustable dual-arm robot base, and drives an arm of a patient to move through two end effectors. The movable space adjustable dual-arm robot base adjusts an operating space of the dual-arm rehabilitation robot. The position tracker is used for real-time collecting position and posture information of the arm, and transmitting it to the host computer control center and the interactive display screen. The interactive display screen is used for synchronous operating a game by the position and posture information. The host computer control center is used to store patient information, and is used to provide a quantitative index after evaluating a rehabilitation process of the patient.

### BACKGROUND

### Technical Field

The invention relates to the field of upper limb rehabilitation, and particularly relates to an interactive upper limb rehabilitation training system.

### Description of Related Art

With the improvement of living standards, proportion of aging population continues to increase. Stroke is a disease with high incidence rate in elderly people. Hemiplegia caused by the stroke will greatly affect daily life of a patient. Through systematic and scientific training of an upper limb rehabilitation robot, the patient with hemiplegia can recover his/her range of limb movement, muscle strength, flexibility, etc., and improve his/her daily living ability. Rehabilitation process is currently based on experiences of a rehabilitation therapist and there is a difference even if in training the same patient for the same motion. There is a large error in an evaluation of the patient based on the experiences of the rehabilitation therapist or scale. During the rehabilitation process, the patient tends to be boring, and his/her mood after the illness is relatively low. There may be cases of poor motivation for rehabilitation and noncompliance, which affect the rehabilitation effect.

### SUMMARY

In order to solve the above technical problem, the invention discloses an interactive upper limb rehabilitation training system, which is applied to an exercise of an upper limb rehabilitation training process, and realizes a safe, scientific, interesting and data-oriented upper limb rehabilitation training process.

In order to achieve the above object, technical solutions of the invention are as follows.

The interactive upper limb rehabilitation training system includes an interactive display screen, a host computer control center, a dual-arm rehabilitation robot, a movable space adjustable dual-arm robot base, and a position tracker. The dual-arm rehabilitation robot is mounted on the movable space adjustable dual-arm robot base, and drives an arm of a patient to move through two end effectors. The movable space adjustable dual-arm robot base adjusts an operating space of the dual-arm rehabilitation robot by adjusting a relative position of the dual-arm rehabilitation robot to the patient in a vertical direction and a horizontal direction. The position tracker is mounted on the arm of the patient, and is used for real-time collecting position and posture information of the arm of the patient, and transmitting the collected position and posture information of the arm to the host computer control center and the interactive display screen. The interactive display screen is mounted directly in front of the patient, and is used for synchronous operating a game of the interactive display screen by the position and posture information of the arm obtained by the position tracker. The host computer control center is used for storing patient information, and providing a quantitative index after evaluating a rehabilitation process of the patient according to the position and posture information of the arm.

Further, the position tracker includes an upper arm position tracker and a forearm position tracker. The upper arm position tracker and the forearm position tracker are respectively mounted on an upper arm and a forearm of the patient, and are used for real-time collecting position and posture information of the upper arm and the forearm of the patient, and transmitting the collected position and posture information to the host computer control center and the interactive display screen.

Further, the two end effectors of the dual-arm rehabilitation robot are respectively used to drive the upper arm and the forearm of the patient for passive rehabilitation, active rehabilitation, and active and passive rehabilitation.

Further, the movable space adjustable dual-arm robot base includes a level adjustment plate, a height pole, and a rectangular aluminum profile chassis connected in sequence from top to bottom. The level adjustment plate is fixedly provided with two level adjustable robot arm mounting assemblies. A bottom portion of the aluminum profile chassis is provided with level adjustment support casters and adjust feet. Each of the robot arm mounting assemblies includes a longitudinal height adjustment assembly and a height adjustment member mounting plate connected in sequence from top to bottom. A top end of the longitudinal height adjustment assembly is provided with a level robot arm mounting member.

Further, the longitudinal height adjustment assembly includes a T-shaped longitudinal height adjustment member that connects the level robot arm mounting member, and a longitudinal height adjustment member that is fixed to the height adjustment member mounting plate by bolts. The T-shaped longitudinal height adjustment member is evenly disposed in a plurality of threaded holes along the height direction. The threaded holes are used for connecting the longitudinal height adjustment member by the bolts.

Further, the longitudinal height adjustment member includes a symmetrically disposed L-shaped right longitudinal height adjustment member and a symmetrically disposed L-shaped left longitudinal height adjustment member. The T-shaped longitudinal height adjustment member is fixedly clamped between the right longitudinal height adjustment member and the L-shaped left longitudinal height adjustment member by the bolts.

Further, both sides of the aluminum profile chassis are further connected with a torque support pole prepared by an aluminum profile through a living hinge, and a free end of the torque support pole is provided with the adjust foot.

Further, the level adjustment plate is provided with a plurality of threaded holes in a length direction. The threaded holes are used for connecting the height adjustment member mounting plate.

Further, the height pole includes a pole body. Upper and lower ends of the pole body are respectively fixedly connected to the level adjustment plate and the aluminum profile chassis through a connection plate. Alternatively, the upper or lower end of the pole body is respectively rotationally connected to the level adjustment plate or the aluminum profile chassis through the connection plate and is provided with a locking device.

Further, the patient information includes age, gender, disease condition, muscle strength level, treatment time, rehabilitation change, shoulder joint range of motion, elbow joint range of motion, wrist range of motion. The quantitative index given after the evaluation of the rehabilitation process of the patient specifically refers to the quantitative index given to upper limb range of motion, the muscle strength level, and upper limb coordination based on Fugl-Meyer scales according to the stored patient information.

Compared with the prior art, the invention has the following advantages and technical effects.

The invention realizes the safe, scientific, interesting and data-oriented upper limb rehabilitation training process by providing the interactive upper limb rehabilitation training system comprising the interactive display screen, the host computer control center, the dual-arm rehabilitation robot and the position tracker. At the same time, the operating space of the robot arm is more flexible, the movement of the robot arm is more convenient, the support of the robot arm is more stable, and the space occupied by the robot arm is more economical.

### BRIEF DESCRIPTION OF THE DRAWINGS

FIG. 1 is a schematic diagram of an interactive upper limb rehabilitation training system.

FIG. 1 includes: 1—dual-arm rehabilitation robot, 2—level robot arm mounting member, 3—T-shaped longitudinal height adjustment member, 4—longitudinal height adjustment member, 5—height adjustment member mounting plate, 6—level adjustment plate, 7—height pole, 8—aluminum profile chassis, 9—level adjustment support caster, 10—cross-section cover, 11—torque support pole, 12—adjustable foot, 13—upper arm position tracker, 14—end effector, 15—forearm position tracker, 16—interactive display screen, 17—host computer control center.

### DESCRIPTION OF THE EMBODIMENTS

The invention will be further described below in conjunction with embodiments and accompanying drawing, but is not limited thereto.

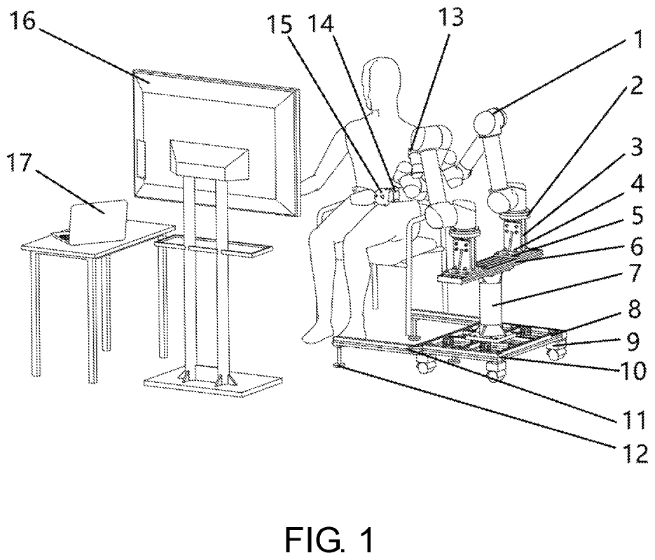

As shown in FIG. 1, an interactive upper limb rehabilitation training system includes an interactive display screen 16, a host computer control center 17, a dual-arm rehabilitation robot 1, a movable space adjustable dual-arm robot base and a position tracker.

The dual-arm rehabilitation robot 1 is mounted on the movable space adjustable dual-arm robot base. Two end effectors 14 of the dual-arm rehabilitation robot 1 are respectively used to drive an upper arm and a forearm of a patient for passive rehabilitation, active rehabilitation and active and passive rehabilitation, thereby realizing a scientific rehabilitation process.

The position tracker includes an upper arm position tracker 13 and a forearm position tracker 15. The upper arm position tracker 13 and the forearm position tracker 15 are respectively mounted on the upper arm and the forearm of the patient, and are used for real-time collecting position and posture information of the upper arm and the forearm of the patient, and transmitting the collected position and posture information to the host computer control center 17 and the interactive display screen 16, thereby realizing real-time monitoring, security protection and a safe rehabilitation process.

The interactive display screen 16 is mounted directly in front of the patient, and is used for synchronous operating a game of the interactive display screen by the position and posture information of the arm obtained by the position tracker, thereby realizing the interaction between the system and the patient, and an interest rehabilitation process.

The host computer control center 17 is a computer for storing patient information such as age, gender, disease condition, muscle strength level, treatment time, rehabilitation change, shoulder joint range of motion, elbow joint range of motion, wrist range of motion, etc. A quantitative index is given to upper limb range of motion, the muscle strength level, and upper limb coordination based on Fugl-Meyer scales according to the stored patient information, thereby realizing a data-oriented rehabilitation process.

The movable space adjustable dual-arm robot base includes a level adjustment plate 6, a height pole 7, and a rectangular aluminum profile chassis 8 connected in sequence from top to bottom. The level adjustment plate 6 is provided with two level adjustable robot arm mounting assemblies. A bottom portion of the aluminum profile chassis 8 is provided with level adjustment support casters 9 and adjustable feet 12. The level adjustable robot arm mounting assemblies include a longitudinal height adjustment assembly and a height adjustment member mounting plate 5 connected in sequence from top to bottom. A top end of the longitudinal height adjustment assembly is provided with a level robot arm mounting member 2. The support of the level adjustment support caster 9 and the adjustable foot 12 allows the base unit to adapt to different ground conditions and to ensure the level of the base.

The longitudinal height adjustment assembly includes a T-shaped longitudinal height adjustment member 3 that connects the level robot arm mounting member 2, and a longitudinal height adjustment member that is fixed to the height adjustment member mounting plate 6 by bolts. The T-shaped longitudinal height adjustment member 3 is evenly disposed in a plurality of threaded holes along a height direction. The threaded holes are used for connecting the longitudinal height adjustment member by the bolts. The longitudinal height adjustment member 4 includes two symmetrically disposed L-shaped longitudinal height adjustment members. The T-shaped longitudinal height adjustment member 3 is fixedly clamped between the two symmetrically disposed L-shaped longitudinal height adjustment members by the bolts. The selection of the threaded holes can be adjusted in the height direction with respect to the longitudinal height adjustment member 4, thereby realizing the change of the dual-arm robot operating space in a vertical direction.

Both sides of the aluminum profile chassis 8 are further connected with a torque support pole 11 prepared by an aluminum profile by a living hinge, and a free end of the torque support pole 11 is provided with the adjustable foot 12. The torque support pole prevents the base and the robot arm from falling over, and saves space after rotating around the hinge.

The torque support pole 11 is made of the aluminum profile, and the aluminum profile chassis 8 is mainly formed by connecting a plurality of aluminum profiles, corner pieces and bolts. A cross-section cover 10 is provided on a cross section of the aluminum profile.

The level adjustment plate 6 is mainly composed of a plurality of aluminum profiles arranged in parallel with each other. The level adjustment plate 6 is provided with a plurality of threaded holes in a horizontal direction. The threaded holes are used for connecting the height adjustment member mounting plate 5. The relative position of the height adjustment member mounting plate 5 and the level adjustment plate 6 in the horizontal direction can be adjusted by the selection of the threaded holes, thereby realizing the change of the dual-arm robot operating space in the horizontal direction.

The height pole 7 includes a pole body, and upper and lower ends of the pole body are respectively fixedly connected to the level adjustment plate 6 and the aluminum profile chassis 8 through a connection plate.

In a possible embodiment, the upper or lower end of the pole body is rotationally connected to the connection plate and provided with a locking device. The horizontal adjustment plate 6 can be rotated and locked in a desired position when needed.

In the interactive upper limb rehabilitation training system provided by the above embodiments, the dual-arm rehabilitation robot 1 is mounted on the movable space adjustable dual-arm robot base. The dual-arm robot drives the upper arm of the patient to exercise, and realizes passive rehabilitation, active and passive rehabilitation and active rehabilitation of the patient. The whole system realizes a safe, scientific, interesting and data-oriented upper limb rehabilitation training process. At the same time, the operating space of the robot arm is more flexible, the movement of the robot arm is more convenient, the support of the robot arm is more stable, and the space occupied by the robot arm is more economical.

Variations and modifications of the above-described embodiments may also be made by those skilled in the art in light of the above disclosure. Therefore, the invention is not limited to the detailed embodiments disclosed and described herein, and the modifications and variations of the invention are intended to fall within the scope of the appended claims.

1. An interactive upper limb rehabilitation training system, comprising an interactive display screen, a host computer control center, a dual-arm rehabilitation robot, a movable space adjustable dual-arm robot base and a position tracker; wherein
the dual-arm rehabilitation robot is mounted on the movable space adjustable dual-arm robot base, and is adapted to drive an arm of a patient to move through two end effectors;
the movable space adjustable dual-arm robot base adjusts an operating space of the dual-arm rehabilitation robot by adjusting a relative position of the dual-arm rehabilitation robot to the patient in a vertical direction and a horizontal direction;
the position tracker is mounted on the arm of the patient, and is used for real-time collecting position and posture information of the arm of the patient, and transmitting the collected position and posture information of the arm to the host computer control center and the interactive display screen;
the interactive display screen is mounted directly in front of the patient, and is used for synchronous operating a game of the interactive display screen by the position and posture information of the arm obtained by the position tracker; and
the host computer control center is used for storing patient information, and providing a quantitative index after evaluating a rehabilitation process of the patient according to the position and posture information of the arm.

2. The interactive upper limb rehabilitation training system according to claim 1, wherein the position tracker comprises an upper arm position tracker and a forearm position tracker; the upper arm position tracker and the forearm position tracker are respectively mounted on an upper arm and a forearm of the patient, and are used for real-time collecting position and posture information of the upper arm and the forearm of the patient, and transmitting the collected position and posture information to the host computer control center and the interactive display screen.

3. The interactive upper limb rehabilitation training system according to claim 1, wherein the two end effectors of the dual-arm rehabilitation robot are respectively used to drive an upper arm and a forearm of the patient for passive rehabilitation, active rehabilitation, and active and passive rehabilitation.

4. The interactive upper limb rehabilitation training system according to claim 1, wherein the movable space adjustable dual-arm robot base comprises a level adjustment plate, a height pole, and a rectangular aluminum profile chassis connected in sequence from top to bottom; the level adjustment plate is fixedly provided with two level adjustable robot arm mounting assemblies; a bottom portion of the aluminum profile chassis is provided with level adjustment support casters and adjustable feet; each of the robot arm mounting assemblies comprises a longitudinal height adjustment assembly and a height adjustment member mounting plate connected in sequence from top to bottom; a top end of the longitudinal height adjustment assembly is provided with a level robot arm mounting member.

5. The interactive upper limb rehabilitation training system according to claim 4, wherein the longitudinal height adjustment assembly comprises a T-shaped longitudinal height adjustment member that connects the level robot arm mounting member, and a longitudinal height adjustment member that is fixed to the height adjustment member mounting plate by bolts; the T-shaped longitudinal height adjustment member is evenly disposed in a plurality of threaded holes along the height direction; the threaded holes are used for connecting the longitudinal height adjustment member by the bolts.

6. The movable space adjustable dual-arm robot base unit according to claim 5, wherein the longitudinal height adjustment member comprises a symmetrically disposed L-shaped right longitudinal height adjustment member and a symmetrically disposed L-shaped left longitudinal height adjustment member; the T-shaped longitudinal height adjustment member is fixedly clamped between the right longitudinal height adjustment member and the left longitudinal height adjustment member by the bolts.

7. The interactive upper limb rehabilitation training system according to claim 4, wherein both sides of the aluminum profile chassis are connected with a torque support pole prepared by an aluminum profile through a living hinge, and a free end of the torque support pole is provided with the adjustable foot.

8. The interactive upper limb rehabilitation training system according to claim 4, wherein the level adjustment plate is provided with a plurality of threaded holes in a length direction; the threaded holes are used for connecting the height adjustment member mounting plate.

9. The interactive upper limb rehabilitation training system according to claim 4, wherein the height pole comprises a pole body; upper and lower ends of the pole body are respectively fixedly connected to the level adjustment plate and the aluminum profile chassis through a connection plate; alternatively, the upper or lower end of the pole body is respectively rotationally connected to the level adjustment plate or the aluminum profile chassis through the connection plate and is provided with a locking device.

10. The interactive upper limb rehabilitation training system according to claim 1, wherein the patient information comprises age, gender, disease condition, muscle strength level, treatment time, rehabilitation change, shoulder joint range of motion, elbow joint range of motion, wrist range of motion; the quantitative index given after the evaluation of the rehabilitation process of the patient specifically refers to the quantitative index given to upper limb range of motion, the muscle strength level, and upper limb coordination based on Fugl-Meyer scales according to the stored patient information.
